(19) United States
(12) Patent Application Publication
(10) Pub. No.: US 2016/0256181 A1
(43) Pub. Date: Sep. 8, 2016

(54) MORCELLATOR CONCEPT FOR TONSILLECTOMY

(71) Applicant: COVIDIEN LP, MANSFIELD, MA (US)

(72) Inventors: JAMES D. ALLEN, IV, BROOMFIELD, CO (US); ROBERT M. SHARP, BOULDER, CO (US); GARY M. COUTURE, WARD, CO (US); CASSANDRA LATIMER, THORNTON, CO (US)

(21) Appl. No.: 14/925,432

(22) Filed: Oct. 28, 2015

Related U.S. Application Data
(60) Provisional application No. 62/129,086, filed on Mar. 6, 2015.

(51) Int. Cl. A61B 17/26; A61B 17/32; A61B 18/14

(52) U.S. Cl. CPC A61B 17/26; A61B 18/149; A61B 17/32002; A61B 2018/00327

(57) ABSTRACT

A morcellator includes an elongated tube, a shaft, and a blade. The elongated tube has proximal and distal ends and defines a channel therethrough. The shaft is positioned within the channel and has a distal end portion and has a longitudinal axis defined therealong. The blade is positioned on the distal end portion of the shaft and is configured to rotate about the longitudinal axis of the shaft and to deliver electrosurgical energy to tissue.

### CROSS REFERENCE TO RELATED APPLICATION

The present application claims the benefit of and priority to U.S. Provisional Application Ser. No. 62/129,086, filed on Mar. 6, 2015, the entire contents of which are incorporated herein by reference.

### BACKGROUND

1. Technical Field

The present disclosure relates to tissue morcellation and, more specifically, to tissue morcellators, morcellation systems, and tissue morcellation methods for use during tonsillectomy procedures.

2. Discussion of Related Art

Tonsillectomies are commonly performed surgical procedures in the United States and elsewhere, with an estimated 340,000 procedures performed in the United States each year. Tonsillectomies are indicated for recurrent infection of the tonsils and surrounding tissues and/or airway obstruction caused by the tonsils. Post-operatively, tonsillectomies are painful due to the resultant exposure of unprotected oropharyngeal nerve and surrounding muscle tissue. The healing process takes between two to three weeks for the injured tissue to become overgrown with new epithelial/mucous membrane tissue. This period is typically characterized by considerable pain, dehydration, and weight loss due to difficulty in swallowing as well as occasional hemorrhaging.

While suction electrocoagulation for removal of tonsils is an effective method with minimal blood loss (e.g. blood vessels are electrocoagulated as they are encountered), the procedure has several important limitations. First the coagulation may cause trauma to surrounding and subjacent tissues beyond the point of contact. This causes unwanted tissue necrosis and may lead to late post-operative bleeding. Also, suction electrocoagulation devices have limited dissecting capabilities. Nonetheless, there is a tendency to use such electrocoagulation devices to cut through adherent tissue which leads to even greater tissue trauma. The other option is to switch back and forth between using a suction electrocoagulation device and a dissecting instrument. Such switching between instruments prolongs the surgery and the time the patient must remain under anesthetic, permits additional hemorrhaging during instrument changes, and decreases efficiency.

Another method for the removal of tonsils involves the use of bipolar electrocoagulating forceps. With bipolar electrocoagulation, tissue damage is limited to the tissue between the tines of the bipolar coagulator. The bipolar electrocoagulating forceps is designed principally for coagulation. If suction is needed, which is not uncommon, another instrument must be introduced into the operating field.

### SUMMARY

There remains a need for new devices, systems, and method for performing tonsillectomies that precisely remove the tonsil tissue while reducing bleeding and trauma to surrounding tissue.

In an aspect of the present disclosure, a morcellator includes an elongated tube, a shaft, and a blade. The elongated tube has proximal and distal ends and defines a channel therethrough. The shaft is positioned within the channel and has a distal end portion. The shaft defines a longitudinal axis. The blade is positioned on the distal end portion of the shaft. The blade is configured to rotate about the longitudinal axis of the shaft and to deliver electrosurgical energy to tissue.

In some aspects, the elongated tube may define a longitudinal axis that is parallel to the longitudinal axis of the shaft. The elongated tube may be curved along its longitudinal axis. Additionally, the elongated tube may be flexible along its longitudinal axis.

In particular aspects, the elongated tube is configured to couple to a suction source that provides suction at a distal end through the channel.

In another aspect of the present disclosure, a morcellation system includes a vacuum source, an electrosurgical energy source, and a morcellator. The morcellator includes an elongated tube, a shaft, and a blade. The elongated tube has proximal and distal ends and defines a channel through. The shaft is positioned within the channel and has a distal end portion. The shaft defines a longitudinal axis. The blade is positioned on the distal end portion of the shaft. The blade is configured to rotate about the longitudinal axis of the shaft and to deliver electrosurgical energy to tissue.

In some aspects, the morcellation system includes a motor that is coupled to a proximal end portion of the shaft to selectively rotate the blade. The elongated tube may include an electrode that is disposed on the inner surface of the distal end of the elongated tube adjacent the blade. The electrode may be configured to return electrosurgical energy from the blade to the electrosurgical energy source. The electrode may be a ring electrode that is disposed about the inner surface of the elongated tube. The morcellator may include a conductor extending from the electrode to the proximal end of the elongated tube that electrically connects the electrode to the electrosurgical energy source. The conductor may be disposed within a wall of the elongated tube.

In certain aspects, the shaft is constructed of a conductive material and electrically connects the electrosurgical energy source to the blade. The blade may be constructed of a conductive material.

In particular aspects, the morcellation system includes a return pad that is in electrical communication with the electrosurgical energy source. The return pad may be configured to return electrosurgical energy from the blade to the electrosurgical energy source.

In some aspects, the morcellation system includes an impedance based monitoring system that is configured to adjust the supply of electrosurgical energy from the electrosurgical energy source in response to an impedance of tissue adjacent the blade.

In another aspect of the present disclosure, a method of morcellating tissue includes positioning a distal end of a morcellator adjacent tissue, activating a suction control of the morcellator to provide suction through a channel defined through the morcellator to draw tissue into the distal end of the morcellator, rotating a blade positioned within the channel adjacent a distal end of the morcellator to morcellate tissue drawing into the distal end of the morcellator, and delivering electrosurgical energy to tissue drawn into the distal end of the morcellator within the blade.

In some aspects, rotating the blade includes activating a motor to rotate a shaft. The blade may be coupled to a distal end portion of the shaft.

In certain aspects, delivering electrosurgical energy to tissue drawn into the distal end of the morcellator with the blade includes activating an electrosurgical energy source to supply energy to the blade. Delivering electrosurgical energy to tissue drawn into the distal end of the morcellator with the blade includes returning electrosurgical energy to the electrosurgical energy source through an electrode that is disposed on the inner surface of the morcellator adjacent the blade.

In particular aspects, the method includes adjusting the electrosurgical energy delivered to tissue based on a detected impedance of the tissue.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a morcellation system in accordance with the present disclosure;

FIG. 2 is a cross-sectional view taken along the section line 2-2 of FIG. 1;

FIG. 3 is a perspective view of another morcellation system in accordance with the present disclosure;

FIG. 4 is side view of a cutaway of a patient and the morcellation system of FIG. 1 during a tonsillectomy procedure in accordance with the present disclosure; and

FIG. 5 is a schematic view of a medical work station provided in accordance with the present disclosure.

### DETAILED DESCRIPTION

Embodiments of the present disclosure are described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

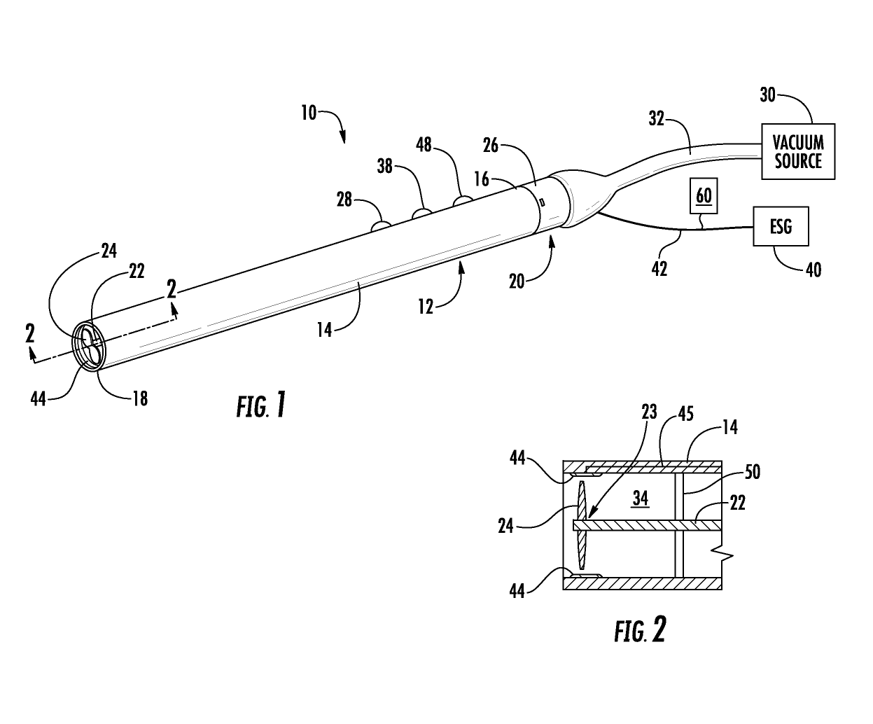

Referring to FIGS. 1 and 2, a morcellation system 10 is provided in accordance with the present disclosure and includes a morcellator 12, a rotary mechanism 20, a vacuum source 30, and an electrosurgical energy source 40. The morcellator 12 includes an elongated tube 14 having a proximal end 16 and a distal end 18. The elongated tube 14 defines a channel 34 between the proximal and distal ends 16, 18.

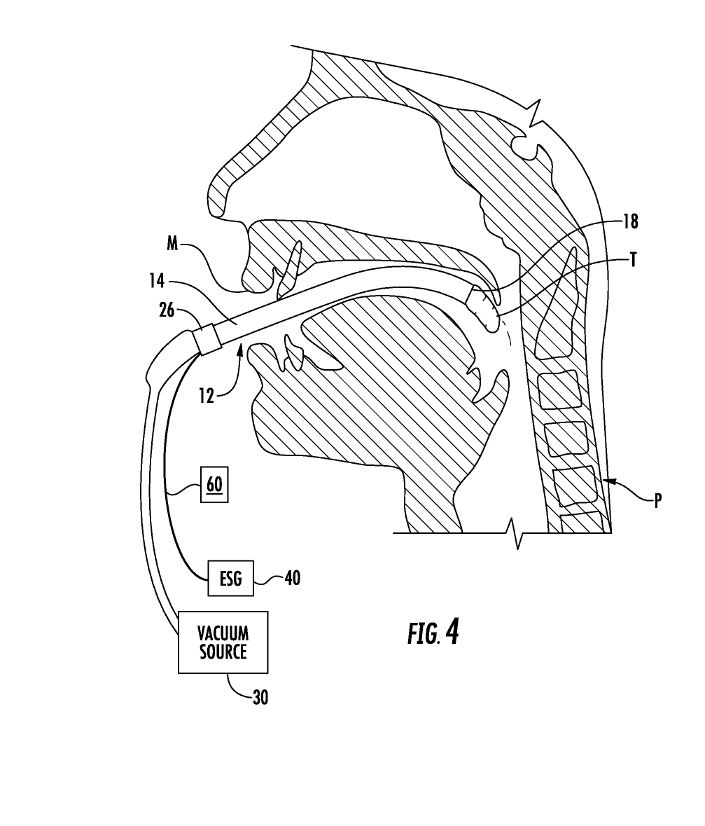

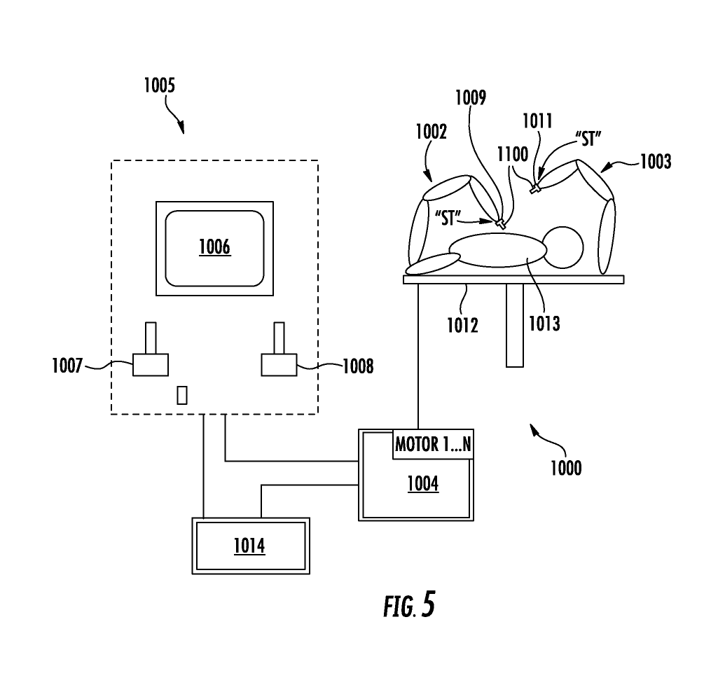

The elongated tube 14 is made of a non-conductive, biocompatible material and is rigid about a longitudinal axis thereof such that the elongated tube 14 maintains a substantially circular cross-section in a plane transverse to the longitudinal axis. The elongated tube 14 may be semi-rigid along the longitudinal axis such that the distal end 18 deflects from the longitudinal axis of during a surgical procedure as detailed below. The elongated tube 14 may also be linear (FIG. 1) or curved (FIG. 4) along the longitudinal axis. The elongated tube 14 includes supports 50 (FIG. 2) positioned within the channel to rotatably support and position the shaft 22 on the longitudinal axis of the elongated tube 14.

The rotary mechanism 20 includes shaft 22, a blade 24, and a motor 26. The motor 26 is positioned at the proximal end 16 of the elongated tube 14. The shaft 22 extends distally from the motor 26 to a distal end portion 23 thereof. As shown in FIG. 2, the distal end portion 23 of the shaft 22 is positioned within the channel 34 of the elongated tube 14 adjacent the distal end 18 of the elongated tube 14; however, it is within the scope of this disclosure that the distal end portion 23 of the shaft 22 may extend beyond the distal end 18 of the elongated tube 14 or that the distal end portion 23 of the shaft 22 is in a common plane with the distal end 18 of the elongated tube 14.

The blade 24 is coupled to the distal end portion 23 of the shaft 22 adjacent the distal end 18 of the elongated tube 14. The motor 26 is operatively associated with the shaft 22 to rotate the blade 24 about a longitudinal axis of the shaft 22 when the motor 26 is activated. The motor 26 is coupled to the proximal end 16 of the elongated tube 14 and seals proximal end 16 of the elongated tube 14. In addition, the motor 26 may permit suction and/or morcellated tissue to pass through the motor 26 to the vacuum source 30 as detailed below. The motor 26 may be coupled to an external power source (e.g., a power outlet), draw energy from the electrosurgical energy source 40, or include a portable power source (e.g., a battery pack) that provides energy to selectively rotate the blade 24 when the motor 26 is activated. The elongated tube 14 may include a motor control 28 positioned adjacent the proximal end 16 of the elongated tube 14 to selectively activate the motor 26 by controlling the delivery of energy to the motor 26.

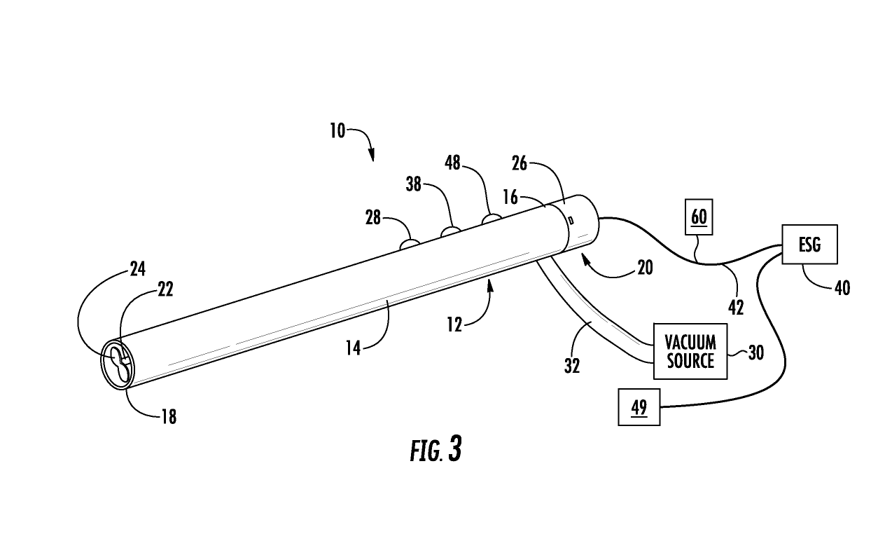

The vacuum source 30 provides suction through the channel 34 defined by the elongated tube 14. As shown, the vacuum source 30 is coupled to the motor 26 by a lumen 32 to provide suction through the channel 34. The lumen 32 is positioned over a proximal end of the motor 26 in sealing relationship therewith to provide suction through the channel 34. The motor 26 may include a passage (not shown) extending proximally therefrom which is coupled to the lumen 32 to provide suction through the channel 34. Alternatively, as shown in FIG. 3, the vacuum source 30 may be connected to the elongated tube 14 distal to the motor 26 to provide suction through the channel 34. The vacuum source 30 is selectively activated to provide suction through the channel 34. The elongated tube 14 may include a vacuum control 38 positioned adjacent the proximal end 16 of the elongated tube 14 to selectively activate the vacuum source 30.

The electrosurgical energy source 40 is operatively associated with the blade 24 to deliver electrosurgical energy to tissue adjacent the blade 24. The electrosurgical energy source 40 may be an electrosurgical generator (ESG) positioned within a surgical theater and coupled to the morcellator 12 by a cable 42. Additionally or alternatively, the cable 42 may be coupled to the motor 20 or the elongated tube 14 to supply electrosurgical energy to the blade 24. The elongated tube 14 may include an energy control 48 positioned adjacent the proximal end 16 of the elongated tube 14 to selectively activate the electrosurgical energy source 40 to supply electrosurgical energy to the blade 24.

In addition, the electrosurgical energy source 40 may include an impedance based monitoring system 60 that is configured to detect the impedance of tissue as the blade 24 delivers electrosurgical energy to the tissue. The impedance based monitoring system 60 may be configured to adjust the electrosurgical energy supplied to the blade 24 by the electrosurgical energy source 40 in response to the detected impedance of the tissue as detailed below.

The blade 24 is configured to deliver electrosurgical energy to tissue positioned within the distal end 18 of the elongated tube 14. The blade 24 may be constructed entirely of a conductive material. The blade 24 may alternatively be formed from a non-conductive material with a conductive coating over at least a portion of its outer surface to deliver electrosurgical energy to tissue.

The shaft 22 may be formed of a conductive material and electrically connected to the electro surgical energy source 40 at its proximal end portion 16 and electrically coupled to the blade 24 within the distal end portion 23. The shaft 22 may be coated with a dielectric material along a portion of its length. The shaft 22 may be formed of a non-conductive material and include a conductor (not shown) disposed on or within the shaft 22 to electrically couple the blade 24 to the electrosurgical energy source 40.

With particular reference to FIG. 2, the elongated tube 14 includes an electrode 44 positioned on an inner surface of the elongated tube 14 and longitudinally aligned with the blade 24. In such embodiments, the blade 24 and the electrode 44 deliver electrosurgical energy to tissue in a bipolar manner. The electrode 44 is in electrical communication with the electrosurgical energy source 40 to return electrosurgical energy delivered to tissue by the blade 24 to the electrosurgical energy source 40. The electrode 44 may be coupled to a conductor 45 disposed on or within the inner surface of the elongated tube 14 to electrically connect the electrode 44 to the electrosurgical energy source 40. The function of the blade 24 and the electrode 44 may be reversed such that the electrode 44 delivers electrosurgical energy to tissue and the blade 24 returns the electrosurgical energy to the electrosurgical energy source 40. The electrode 44 is shown as a ring about the inner surface of the elongated tube 14 adjacent the distal end thereof; however, it is contemplated that the electrode 44 may be a disposed about only a portion of the inner surface of the elongated tube 14 and may include one or more portions that are each disposed about a portion of the inner surface of the elongated tube 14.

Referring to FIG. 3, the blade 24 of the morecellator 12 may function in a monopolar manner. In such embodiments, the electrosurgical energy is delivered to tissue by the blade 24 and returns to the electrosurgical energy source 40 through a return pad 49 in contact with a patient.

With reference to FIG. 4, the morcellation system 10 is used to perform a tonsillectomy procedure. To perform a tonsillectomy, the morcellator 12 is inserted through the mouth “M” of a patient “P” until the distal end 18 of the morcellator 12 is positioned adjacent a tonsil “T” of the patient “P.” The elongated tube 14 of the morcellator 12 may be curved along the longitudinal axis and/or may be flexible along the longitudinal axis to facilitate insertion into and through the mouth “M” of the patient. With the distal end 18 of the morcellator adjacent the tonsil “T,” the vacuum source 30 is activated to provide suction through the channel 34 (FIG. 2) of the morcellator 12 to draw the tonsil “T” into the distal end 18 of the morcellator 12. The suction may be calibrated to draw tonsil tissue, typically light, spongy tissue, into the distal end 18 of the morcellator 12 while leaving muscle, typically more dense tissue, outside of the distal end 18 of the morcellator 12.

As the tonsil “T” is drawn into the distal end 18 of the morcellator 12, the motor 26 is activated to rotate the blade 24 (FIG. 2) to morcellate the tonsil “T” as the tonsil “T” is drawn into the distal end 18 of the morcellator 12. In addition, as the tonsil “T” is drawn into the distal end 18 of the morcellator 12, the electrosurgical energy source 40 is activated to supply electrosurgical energy to the blade 24 such that the blade 24 delivers electrosurgical energy to the tonsil “T” in contact with and adjacent the blade 24. The electrosurgical energy delivered by the blade to the tonsil “T” coagulates and/or cauterizes the tonsil “T” as the tonsil “T” is being morcellated by the blade 24 to reduce bleeding or hemorrhaging during the tonsillectomy procedure. During delivery of the electrosurgical energy, the elongated tube 14, constructed of a non-conductive material, insulates tissue surrounding the tonsil “T” from the electrosurgical energy. In addition, the elongated tube 14 may also insulate tissue surrounding the tonsil “T” from heating as electrosurgical energy is delivered to the tonsil “T.” This shielding of surrounding tissue may shorten the recovery time of the patient after the tonsillectomy procedure, may reduce necrosis of surrounding tissue during and after the tonsillectomy procedure, and/or may reduce the bleeding or hemorrhaging during the tonsillectomy procedure.

When the tonsil “T” is removed the morcellator 12 is removed through the mouth “M” of the patient “P” to complete the tonsillectomy procedure. Individual vacuum, motor, and energy controls 28, 38, 48 may be utilized to independently activate the vacuum source 30, the motor 26, and the electrosurgical energy source 40, respectively, or a common activation control (not explicitly shown) may be utilized to selectively activate one or more of the vacuum source 30, the motor 26, and the electrosurgical energy source 40 simultaneously.

In embodiments where the morcellator 12 is functioning in a bipolar manner, the electrosurgical energy delivery is limited to tissue positioned between the blade 24 and the electrode 44 positioned on the inner wall of the morcellator 12.

Additionally or alternatively, as the blade 24 delivers electrosurgical energy to tonsil tissue, the impedance based monitoring system 60 detects changes in the impedance of the tonsil tissue and adjusts the electrosurgical energy being supplied by the electrosurgical energy source 40 to reduce potential damage to surrounding tissue and to increase the effectiveness of the cutting and/or coagulating of the blade 24. The impedance based monitory system 60 may adjust the frequency and/or the intensity of the electrosurgical energy being supplied by the electrosurgical energy source 40. This adjustment of the electrosurgical energy may enhance the effectiveness of the tonsillectomy procedure, may reduce the time and thus the cost of the tonsillectomy procedure, may shorten the recovery time of the patient after the tonsillectomy procedure, may reduce necrosis of surrounding tissue during and after the tonsillectomy procedure, and/or may reduce the bleeding or hemorrhaging during the tonsillectomy procedure.

While the use of the morcellation system 10 is detailed herein for use during a tonsillectomy procedure, it is contemplated that the morcellation system 10 may be used in a variety of surgical procedures utilizing an elongated morcellator where limiting trauma to surrounding tissue is desired. These procedures may include, but are not limited to, polypectomy, hysteroscopy resection, functional endoscopic sinus surgery, prostatectomy, or other resection procedures in close proximity to nerves or other critical structures.

The morcellation system 10 disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

Referring to FIG. 5, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A morcellator, comprising:
an elongated tube having proximal and distal ends and defining a channel therethrough;
a shaft positioned within the channel having a distal end portion and having a longitudinal axis defined therealong; and
a blade positioned on the distal end portion of the shaft, the blade configured to rotate about the longitudinal axis of the shaft and to deliver electrosurgical energy to tissue.

2. The morcellator according to claim 1, wherein the elongated tube defines a longitudinal axis parallel to the longitudinal axis of the shaft.

3. The morcellator according to claim 2, wherein the elongated tube is curved along its longitudinal axis.

4. The morcellator according to claim 2, wherein the elongated tube is flexible along its longitudinal axis.

5. The morcellator according to claim 1, wherein the elongated tube is configured to couple to a suction source that provides suction at the distal end through the channel.

6. A morcellation system, comprising:
a vacuum source;
an electrosurgical energy source; and
a morcellator including:
an elongated tube having proximal and distal ends and defining a channel therethrough, the vacuum source in communication with the channel to selectively provide suction through the channel;
a shaft positioned within the channel having a distal end portion and having a longitudinal axis defined therealong; and
a blade positioned on the distal end portion of the shaft, the blade configured to rotate about the longitudinal axis of the shaft and to deliver electrosurgical energy from the electrosurgical energy source to tissue.

7. The morcellation system according to claim 6, further comprising a motor coupled to a proximal end portion of the shaft to selectively rotate the blade.

8. The morcellation system according to claim 6, wherein the elongated tube includes an electrode disposed on the inner surface of the distal end of the elongated tube adjacent the blade, the electrode configured to return electrosurgical energy from the blade to the electrosurgical energy source.

9. The morcellation system according to claim 8, wherein the electrode is a ring electrode disposed about the inner surface of the elongated tube.

10. The morcellation system according to claim 8, wherein the morcellator includes a conductor extending from the electrode to the proximal end of the elongated tube to electrically connect the electrode to the electrosurgical energy source.

11. The morcellation system according to claim 10, wherein the conductor is disposed within a wall of the elongated tube.

12. The morcellation system according to claim 6, wherein the shaft is constructed of a conductive material and electrically connects the electrosurgical energy source to the blade.

13. The morcellation system according to claim 6, wherein the blade is constructed from a conductive material.

14. The morcellation system according to claim 6, further comprising a return pad in electrical communication with the electrosurgical energy source and configured to return electrosurgical energy from the blade to the electrosurgical energy source.

15. The morcellation system according to claim 6, further comprising an impedance based monitoring system configured to adjust the supply of electrosurgical energy from the electrosurgical energy source in response to an impedance of tissue adjacent the blade.

16. A method of morcellating tissue, comprising:
positioning a distal end of a morcellator adjacent tissue;
activating a suction control of the morcellator to provide suction through a channel defined through the morcellator to draw tissue into the distal end of the morcellator;
rotating a blade positioned within the channel adjacent a distal end of the morcellator to morcellate tissue drawn into the distal end of the morcellator; and
delivering electrosurgical energy to tissue drawn into the distal end of the morcellator with the blade.

17. The method according to claim 16, wherein rotating the blade includes activating a motor to rotate a shaft, the blade coupled to a distal end portion of the shaft.

18. The method according to claim 16, wherein delivering electrosurgical energy to tissue drawn into the distal end of the morcellator with the blade includes activating an electrosurgical energy source to supply energy to the blade.

19. The method according to claim 18, wherein delivering electrosurgical energy to tissue drawn into the distal end of the morcellator with the blade includes returning electrosurgical energy to the electrosurgical energy source through an electrode disposed on the inner surface of the morcellator adjacent the blade.

20. The method according to claim 16, further comprising adjusting the electrosurgical energy delivered to tissue based on a detected impedance of the tissue.
